(19) United States
(12) Patent Application Publication
(10) Pub. No.: US 2012/0041458 A1
(43) Pub. Date: Feb. 16, 2012

(54) GASTRIC RING WITH FACETS

(75) Inventor: Pascal Paganon, Lyon (FR)

(73) Assignees: Laparoscopie, Vienne (FR); Compagnie Europeenne d'Etude et de Recherche de Dispositifs pour I'Implantation par

(21) Appl. No.: 13/276,616

(22) Filed: Oct. 19, 2011

Related U.S. Application Data
(63) Continuation of application No. PCT/EP2010/054915, filed on Apr. 15, 2010.

(30) Foreign Application Priority Data
Apr. 21, 2009 (FR) 0952601

(51) Int. Cl. A61B 17/00; B29C 31/00

(52) U.S. Cl. 606/151; 264/299

(57) ABSTRACT

An implantable surgical ring is configured to be placed around a biological organ forming a bag in order to modify the flow area thereof. The ring includes a flexible strip capable of winding around an axis to form a loop around the organ and which has one ventral surface. The flexible strip is preformed such that the ventral surface is curved around the axis and divided into a plurality of contiguous and intersecting facets. The facets are connected to one another by transition areas forming predefined hinging creases with angle coverage with respect to the axis of no more than 5 degrees, each facet covering an angle sector substantially between 15 degrees and 35 degrees.

The present invention pertains to the general field of implantable medical devices and especially to surgical rings, such as sphincters or again gastric rings designed to be implanted around the stomach or the esophagus in the context of treatment of obesity.

The present invention relates more particularly to an implantable surgical ring designed to be placed around a biological organ constituting a pouch or a conduit in order to modify the section of passage of said biological organ, said ring comprising a flexible band capable of being wound around an axis (X-X′) to form a loop around the organ, and having a ventral face designed to come into contact with said organ.

There are known ways of treating patients afflicted with severe obesity by implanting a gastric ring inside them, this gastric ring surrounding and gripping the stomach in order to limit the intake of food.

Such rings can be placed around the intact stomach, to perform either a direct gastroplasty or a subsequent by-pass operation during which the surgeon will have preliminarily performed a surgical remodeling of the stomach in order to create a pouch of reduced dimensions therein.

In the latter case, the ring is aimed at countering the expansion of the surgically formed gastric pouch in order to prevent the human body from adapting to this situation by creating a sort of new stomach upstream to the previous one and thus reducing the therapeutic efficiency of the operation.

Although they generally give satisfaction, the prior art rings sometimes suffer from certain drawbacks.

In particular, the implementation of such rings generally has an effect of creating one or more folds, by flexion, on the ventral surface of the ring which comes into contact with the organ.

Now, the random appearance of these occasionally very pronounced folds may have give rise to excessive constriction on the constituent material of the ring and therefore make it work under fatigue with the risk of damaging it or even breaking it locally.

Naturally, this kind of failure of a ring may have major repercussions as regards its therapeutic effect, especially if said ring is provided with an inflatable pouch designed to adjust the gripping force that said ring exerts on the biological organ.

In addition, the formation of folds may lead to an unequal distribution of the gripping force exerted by the ring on the organ, or even lead to the pinching of the wall of said organ, thus causing discomfort or pain to the patient and even in certain cases prompting necrosis of the tissues.

The document WO2004/019671 describes a gastric ring provided with an inflatable balloon having several chambers. At its contact surface with the stomach, the balloon has several facets laid out substantially in a circle. These facets are separated by notches.

The document US2007/0249893 describes a cuff to treat urinary incontinence. The cuff forms a sphincter controlled so as to obtain a gripping of greater or lesser extent on the biological conduit that passes through it. The cuff has a balloon formed by several small balloons separated by deep notches presenting non-inflatable elements.

The document FR2921822 describes a gastric ring. This ring has flexible tabs designed to prevent the ring from sliding relatively to the stomach. The ring has an inflatable structure forming several chambers provided with facets for contact with the stomach. The chambers are separated by extension elements.

The objects assigned to the present invention are therefore aimed at overcoming the drawbacks listed here above and proposing a novel implantable surgical ring capable of providing efficient and atraumatic gripping of a biological organ, while at the same time having long service life.

Another object assigned to the invention is aimed at proposing a novel, implantable surgical ring that has a particularly simple and light structure and costs little to manufacture.

Another object assigned to the invention is aimed at proposing a novel, versatile, adjustable and implantable surgical ring.

Another object assigned to the invention is aimed at proposing a novel implantable surgical ring having predictable, controllable and reproducible behavior.

Another object assigned to the invention is aimed at proposing an implantable surgical ring whose implanting is made easier.

Yet another object assigned to the invention is aimed at proposing a novel implantable surgical ring that provides homogenous gripping of the biological organ.

Finally, another object assigned to the invention is aimed at proposing a novel method for manufacturing an implantable surgical ring that can be used, by means of simple and low-cost tooling, to obtain a surgical ring that meets the above-mentioned requirements.

The objects assigned to the invention are achieved by means of an implantable surgical ring to be placed around a biological organ, forming a pouch or a conduit in order to modify the section of passage of said biological organ, said ring comprising a flexible band that is capable of getting wound around an axis (X-X′) to form a loop around the organ and that has a ventral face designed to come into contact with said organ, said ring being characterized in that said flexible band is preformed in such a way that the ventral face is curved around said axis (X-X′) and divided into a plurality of contiguous and secant facets which are connected to one another by transition zones forming predefined hinging folds whose angular coverage relatively to the axis (X-X′) is smaller than or equal to 5°, preferably smaller than or equal to 3°, and in a particularly preferable way, smaller than or equal to 1°. Each facet covers an angular sector ranging substantially from 15° to 35°.

The objects assigned to the invention are achieved by means of a method for manufacturing an implantable surgical ring to be placed around a biological organ, forming a pouch or a conduit in order to modify the section of passage of said biological organ, said method comprising a step (a) for making a flexible band that is capable of getting wound around an axis (X-X′) to form a loop around the organ and having a ventral face designed to come into contact with said organ, said method being characterized in that it comprises a step (b) for forming the ventral face, the step comprising a sub-step (b1) for preforming under flexion during which the ventral face is given a shape curved around said axis (X-X′) and a sub-step (b2) of division during which there are formed, on said ventral face, a plurality of contiguous and secant facets that are connected to one another by transition zones forming predefined hinging folds whose angular coverage relatively to the axis (X-X′) is smaller than or equal to 5°, preferably smaller than or equal to 3°, and in a particularly preferable way, smaller than or equal to 1°. Each facet covers an angular sector ranging substantially from 15° to 35°.

Other objects, features and advantages of the present invention shall appear in greater detail from the following description, as well as from the appended drawings, given purely by way of a non-exhaustive illustration, of which:

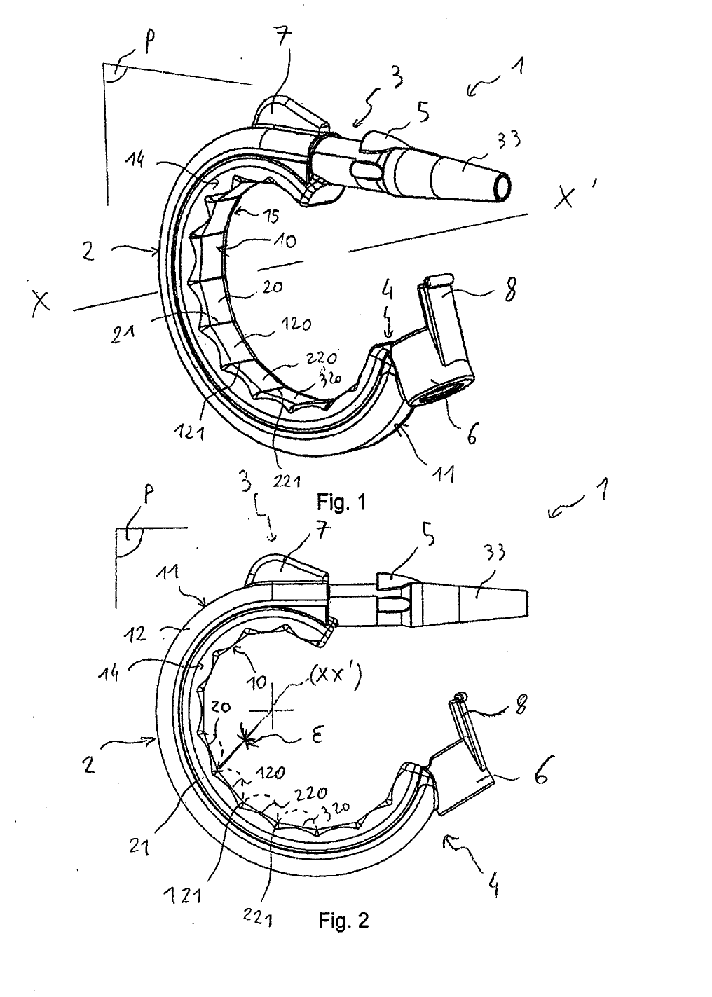

FIG. 1 shows a view in perspective of an implantable surgical ring according to the invention in an open configuration.

FIG. 2 shows a side view of the surgical ring of FIG. 1.

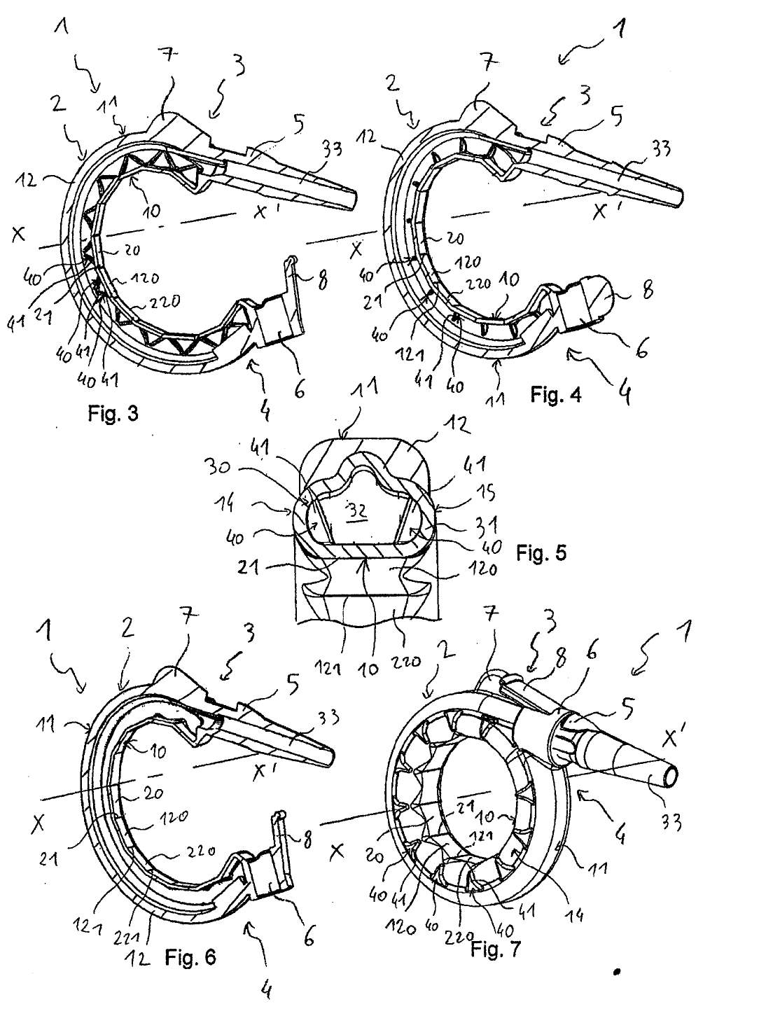

FIG. 3 shows a cut-away perspective view, in the sagittal plane, of an alternative embodiment of a ring such as the one shown in FIGS. 1 and 2.

FIG. 4 is a view similar to that of FIG. 3 of another alternative embodiment of a surgical ring according to the invention.

FIG. 5 is a cross-section view of a part of a ring according to the one shown in FIG. 3.

FIG. 6 is a view similar to that of FIGS. 3 and 4 of a third embodiment of a surgical ring according to the invention.

FIG. 7 is a view in perspective of a fourth alternative embodiment of a surgical ring according to the invention in a closed configuration.

The present invention generally pertains to an implantable surgical ring 1 designed to be placed around a biological organ (not shown) constituting a pouch or a conduit in order to modify the section of passage of said biological organ.

According to one alternative embodiment, said ring 1 can be laid out to form a sphincter designed to regulate the blood flow or to treat urinary or fecal incontinence.

However, preferably, the ring 1 is a gastric ring for the treatment of obesity, which is designed to be positioned on the esophagus or on the stomach.

In a particularly preferred way, the ring 1 is specifically adapted to being placed on the stomach pouch that results from a surgical by-pass operation, in order to counter the post-operational expansion of said stomach pouch and thus constitute a gastric by-pass ring.

According to the invention, the ring 1 has a flexible band 2 which is capable of getting wound around an axis (X-X′) to form a loop around the organ.

Although it is possible, without departing from the framework of the invention, to consider a case where the flexible band 2 forms a sort of open saddle designed to partially surround the concerned organ, for example in a U shape, said flexible band 2 will preferably be long enough to substantially contain the entire perimeter of the biological organ and especially to be closed on itself, preferably substantially at its ends 3, 4 so as to form a closed loop surrounding the organ.

To this end, the flexible band is preferably provided with locking means 5, 6 designed to keep it in a closed configuration, said locking means comprising for example one or more male elements, such as a pin, designed to cooperate, for example by a clip-on process, with a conjugate female element 6 such as a sleeve.

Advantageously, the locking means 5, 6 are reversible so the ring can pass alternately under the control of the practitioner from an open configuration in which its ends 3, 4 are separate and distant, as shown for example in FIGS. 1 and 2, to a closed configuration in which its ends 3, 4 are joined and kept in contact with one another, as shown in FIG. 7.

Naturally, the contour demarcated by the ring 1 according to the invention, around the biological organ, is in no way limited to a particular shape.

However, the ring 1 will be preferably designed so that, when it is in a closed configuration, it adopts the shape of a circular loop with an axis (X-X′).

Besides, the ring 1 may have one or more grasping tabs 7, 8 designed to facilitate the handling of said ring 1, especially when it is being implanted, and/or to enable the actuation of the locking means 5, 6.

As shown in the figures, the flexible band 2 has a ventral face 10 that is designed to come into contact with said organ so as to grip this organ.

Preferably, the flexible band 2 is thus demarcated by said ventral face 10 internally, i.e. as close as possible to the axis (X-X′), and demarcated externally by a dorsal portion 11 opposite said ventral surface 10 and in the zone at the greatest distance from the axis (X-X′).

Preferably, as illustrated especially in FIG. 5, the dorsal portion 11 is formed by a peripheral belt 12 which is preferably substantially non-extensible so as to form a supporting organ whose perimeter is substantially invariant.

Moreover, the flexible band 2 preferably has a first lateral wall 14 and a second lateral wall 15 positioned in either side of the ventral face 10 along the axis (X-X′) and joining said ventral face 10 to the dorsal portion 11.

Advantageously, the shoulder marking the transition between the ventral face 10 and the side faces 14, 15 has a rounded and non-traumatic contour.

According to a preferred characteristic of the invention, the flexible band 2 is preformed so that the ventral face 10 is curved around the axis (X-X′).

Advantageously, giving the flexible band 2 a shape at rest that has a certain curvature which “initiates” the flexion of said flexible band on itself around the axis (X-X′) facilitates the placing of the ring 1 around said organ and especially its closure by bringing its ends 3, 4 closer together.

Naturally, the initial curvature at rest of said flexible band 2 could be freely chosen during manufacture by those skilled in the art, and could be pronounced to a greater or lesser extent.

According to a major characteristic of the invention, the ventral face 10 is divided into a plurality of contiguous and secant facets 20, 120, 220, 320 ( . . . ), that are connected to one another by transition zones 21, 121, 221 ( . . . ) forming predefined hinging folds whose angular coverage relatively to the axis (X-X′) is smaller than or equal to 5°, preferably smaller than or equal to 3°, and in a particularly preferable way smaller than or equal to 1°. Each facet covers an angular sector substantially ranging from 15° to 35°.

The term “angular coverage” designates the angle ε corresponding to the arc formed by a transition zone 21 in a plane P normal to the axis (X-X′) whose vertex is situated on said axis (X-X′) as illustrated in FIG. 2.

Advantageously, the layout of the invention enables the internal contour of the ring 1 to be made discrete at the ventral face 10 in a longitudinal sequence of successive facets 20, 120, 220, 320, preferably situated substantially on the same abscissa relatively to the axis (X-X′) and makes it possible to define sections at the flexible band 2 that are hinged with respect to one another at the transition zones 21, 121 which are particularly narrow, i.e. each of these zones has a particularly narrow or even substantially zero angular coverage e along the perimeter of the flexible band 2.

In a particularly advantageous way, the layout of the invention therefore enables the pre-creation of the preferred folding zones at the transition zones 21, 121, 221 situated at the interface between the facets 20, 120, 220, 320, and therefore makes it possible to control the location and amplitude of formation of the folds when the ring 1 is placed around the stomach.

In this respect, the combination of a curved preform and a division into facets as claimed optimizes the control over the folding zones and limits the fatigue undergone by the constituent material of the flexible band 2 and more particularly the ventral face 10.

For convenience of description, the facets as well as the transition zones shall be referred to in their totality. However, it is perfectly possible, without departing from the framework of the invention, to envisage a case where one or another of the characteristics described could apply to a facet or to a joining zone in particular, or again to the majority or the totality of the facets, and/or the transition zones. Naturally, certain facets and transition zones may have inherent characteristics which distinguish them from the other facets or other transition zones.

Preferably, the transition zones 21, 121, 221 are formed by ridges which correspond to the intersection of the contiguous facets as illustrated in FIGS. 1 to 7.

Preferably, the transition zones 21, 121, 221 are particularly narrow and shallow. Preferably, they are linear and substantially parallel to the axis (X-X′) directly resulting from the edge-to-edge joining of the two facets that are adjacent to them and preferably form an acute angle.

In other words the transition zones preferably, apart from the fillets, amount rectilinear lines joining the first lateral face 14 to the second lateral face 15 and marking the boundary of separation between the successive facets.

Naturally, the number, shape and dimensions of the facets 20, 120, 220, 320 as well as the transition zones that are interposed between these facets can be made to undergo variations without departing from the framework of the invention.

However, the ring 1 of the invention will preferably comprise at least 4 facets, or at least 6 facets or even 12 facets as illustrated in FIGS. 1 and 2.

Each facet 20, 120, 220, 320 could have a relatively large width so as to provide a substantially non-traumatic supporting surface and limit the risks of having the wall of the biological organ pinched.

More particularly, each facet will preferably cover an angular sector measured around the axis (X-X′) and in the plane P, substantially ranging from 15° to 35°.

Moreover, the ventral face 10 will, throughout its length, i.e. between the first end 3 of the flexible band 2 and the second end 4 of this band, preferably show a non-interrupted succession of facets and alternating transition zones.

Preferably, the distribution of the facets will be regular along the ventral face 10. More particularly, said facets should be equally distributed and oriented so that their respective right bisector lines converge toward the axis (X-X′) and intersect substantially at said axis.

According to a preferred alternative embodiment, the facets 20, 120, 220, 320, are substantially identical to one another and in particular may have substantially the same angular coverage and the same longitudinal and transverse dimensions.

Thus, when the ring is in a closed configuration, the ventral face 10 can advantageously form a regular polyhedron whose faces are preferably substantially parallel to the axis (X-X′).

Such a layout may advantageously provide a homogenous gripping of the biological organ.

Preferably, the facets 20, 120, 220, 320, are mounted so as to be radially mobile relatively to the dorsal portion 11 of the flexible band 2.

In other words, the ring 1 is advantageously designed to enable the practitioner to adjust the diameter of its aperture through which said biological organ passes, by notifying the distance between the axis (X-X′) and said facets.

Thus, the surgical ring 1 is advantageously provided with adjusting means which make it possible to adjust the constriction of the biological organ by the flexible band 2 and more particularly the apparent perimeter demarcated by the ventral face 10.

To this end, the facets 20, 120, 220, 320 preferably demarcate one or more inflatable pouches 30 so that they can be driven in radial movement by elastic deformation and/or by folding/unfolding motions, pneumatically or hydraulically, by the addition or withdrawal of fluid to or from the ring 1.

In a particularly preferable way, as illustrated especially in FIGS. 3, 4 and 6, the facets belong to one and the same inflatable pouch 30, in this case a substantially ring-shaped pouch, which extends substantially throughout the length of the flexible band 2, from its first end 3 to its second end 4 and which is transversely segmented by the folds marked by the transition zones.

More particularly, the inflatable pouch 30 can be formed by a substantially tubular membrane 31 made out of a silicone type biocompatible elastomer. Advantageously, the inflatable pouch 30 is provided with only one wall thickness throughout its section and throughout its length.

Preferably, said membrane 31 integrally forms the ventral face 10 and especially the facets as well as the first and the second lateral wall 14, 15 as illustrated in FIG. 5, thus improving its mechanical worthiness and its imperviousness.

Advantageously, the inflatable pouch 30 demarcates a cavity 32 which communicates with a tip 33 enabling the addition or withdrawal of fluid into or from said cavity, for example by means of a catheter connected to a fluid container at a distance (not shown).

In this respect, it can be noted that the membrane 31 can advantageously give the sequence of facets and transition zones a continuous, one-piece structure distinct from the dorsal belt 12 and situated at a distance from this belt.

The inventors have noted that it is possible to obtain an ordered, predictable and reproducible folding of the ring by means of a simple operation for making the facets of the ventral face 10 discrete, for example by means of a ring as shown in FIG. 6.

Thus, through the simple fact of the marking of the fold at the membrane 31 by construction, the marking formed by the folds and more particularly by the preformed ridges on the surface of the ventral face 10 is exactly what is necessary and sufficient to create an well-localized incipient flexion that makes it possible to obtain a controlled and reproducible behavior of the ring.

However, according to a preferred characteristic which may constitute an invention as such, the surgical ring 1 is provided with retaining means 40 which act on the transition zones 21, 121, 221 in order to counter their radial shift when the facets 20, 120, 220, 320 which neighbor them are driven in radial shift.

Indeed, the inventors have been able to establish the fact that the control of the position and uniformity of the folding are improved when elements are provided that are capable of substantially retaining the transition zones and more particularly the ridges at a constant distance from the dorsal portion 11.

More particularly, it proves to be advantageous to provide an element capable of exerting a substantially radial tensile force capable of preventing or at least limiting the centripetal radial shift of said transition zones relatively to that of the free facets as is the case for the alternative embodiments illustrated in FIGS. 3, 4, 5 and 7 in particular.

Preferably, to this end, the retaining means 40, to this end, have one or more tie-rods 41 which mechanically join the transition zones and more particularly the ridges to the dorsal portion 11 of the flexible band 2.

Advantageously, these tie-rods may be internal or external, i.e. positioned inside the inflatable pouch 30 as illustrated in FIGS. 3, 4 and 5 or again outside said pouch 30 as illustrated in FIG. 7.

In either case, they are preferably laid out to permit the free circulation of fluid within the inflatable pouch 30 on either side of the ridges, i.e. in the difference sections demarcated between said ridges and corresponding to the facets.

The tie-rods 41 may be formed by elements having a higher intrinsic rigidity than that of the membrane 31 and especially by tabs integrally joined to the dorsal belt 12 as illustrated in FIG. 7 or again by excess rib-type thicknesses of material preferably integrally joined to the membrane 31.

According to a preferred embodiment, each ridge has at least two matching tie-rods 41 each extending on a lateral face 14, 15 of the flexible band 2 on either side of the ridge as illustrated in FIGS. 5 and 7.

The tie-rods 41 can especially take the form of plates forming ribs which may be positioned either radially, i.e. substantially perpendicularly to the dorsal portion 11 and to the ventral face 10 as shown in FIG. 4, or obliquely as shown in FIG. 3.

In this respect, according to one preferred embodiment illustrated in said FIG. 3, the tie-rods 41 may be laid out in sets of two so as to form triangular structures, each ridge being “braced” by means of two pairs of tie-rods 41 forming a triangle with its base fixed to the dorsal portion 11 and with its vertex coinciding with the ridge.

Besides, according to a preferred characteristic which may constitute an invention as such, the facets 20, 120, 220, 320, are substantially plane when the surgical ring is at rest, i.e. especially when the pouch 30 is not deformed by stresses applied by a fluid under pressure.

Thus, the contour at rest of the ventral face 10, considered in the plane P, will preferably substantially take the form of a broken polygonal line and more particularly a regular polygon, when the ring is in its closed configuration. In other words, the ventral face 10 preferably, when projected into the plane P, forms a regular polygon that can be inscribed within a circle centered on the axis (X-X′), the vertices of the polygon being formed by the projections of the ridges, and the sides of the polygon, corresponding to the chords which connect the vertices situated on said circle to one another, being formed by the projections of the facets.

Advantageously, this particularly compact layout in which each facet is substantially in the prolongation of the previous facet facilitates the introduction of the ring around the organ, especially when it has to be engaged in the retrogastric space.

Moreover, such a geometry which is particularly simple is favorable to the making of the ring 1 by molding.

Naturally, the facets could also have a domed geometry and in particular could get deformed during inflation so as to form domes as shown in dashes in FIG. 2.

In this respect, it can be noted that the geometrical and dimensional characteristics, especially the angular coverage values, as well as the proportions specified here above, are preferably measured when the ring is closed and at rest, but they can advantageously be substantially preserved or at any rate change within specified ranges of values during modifications of configuration prompted by the inflation or deflation of the ring.

Besides, it can be noted that the contour formed by the ventral face 10, although concave with respect to the axis (X-X′), is advantageously devoid of shoulders or recesses especially at the transition zones.

Preferably, the thickness of the membrane 31 is substantially constant throughout the stretch of the ventral face 10 and especially throughout the length of the flexible band, especially at the passage of the transition zones between two successive facets.

This contributes especially to simplifying the molding of said membrane.

Naturally, the present invention is in no way limited to a particular alternative embodiment.

It is perfectly possible to envisage a case where the ring 1 of the invention has a plurality of inflatable pouches, especially ring-shaped ones, staged along the axis (X-X′), or again that the inflatable pouch or pouches have a structure with two-way facets, i.e. a matrix structure divided into facets both lengthwise, i.e. along its perimeter around the axis (X-X′) and heightwise measured in a direction parallel to said axis (X-X′).

The present invention also relates to a method for manufacturing a surgical ring 1 having any one of the characteristics described here above.

Thus, said method preferably comprises a step (a) for making a flexible band 2 which is capable of getting wound around an axis (X-X′) to form a loop around the organ and which has a ventral face 10 designed to come into contact with said organ, said method also comprising a step (b) for forming the ventral face 10, said step (b) for forming the ventral face 10, said step (b) for forming the ventral face 10 comprising a sub-step (b1) for preforming under flexion, during which the ventral face is given an curved shape around said axis (X-X′), and a sub-step (b2) of division during which there is formed, on said ventral face 10, a plurality of contiguous and secant facets 20, 120, 220, 320 on said ventral face 10, these facets being connected to one another by transition zones 21, 121, 221 forming predefined hinging folds whose angular coverage relatively to the axis (X-X′) is smaller than or equal to 5°, preferably smaller than or equal to 3° and in a particularly preferred way, smaller than or equal to 1°.

Preferably, the preforming sub-step (b1) is used to give the flexible band 2 a pronounced curvature so that the ring 1 at rest has a substantially C-shaped profile which spontaneously covers at least 180° or even at least 225° around the axis (X-X′).

Preferably, said sub-step (b-2) of division is carried out by contact molding of a polyhedral core which has a succession of plane faces corresponding to negative imprints of the facets.

More particularly, said polyhedral core could take the form of a polyhedron with an axis (X-X′) whose side faces will be substantially regular, plane and parallel to said axis (X-X′), and are inscribed advantageously in a circular cylinder with an axis (X-X′).

Advantageously, the ring 1 according to the invention can therefore be made by means of tooling that is particularly simple to machine and is therefore a low-cost tool entailing no problems of demolding, thus simplifying its manufacture and limiting the discard rate.

The working of a surgical ring 1 according to the invention shall now be described in greater detail.

The practitioner first of all introduces a surgical ring beneath the skin after having made an incision.

To this end he or she works by coelioscopy and introduces the ring 1 through a cannula.

To this end, the practitioner can advantageously straighten the flexible band 2 by force, using its intrinsic elasticity, to temporarily give it a substantially rectilinear shape.

When the ring is in the organism, it tends to spontaneously recover its curved shape by shape memory.

The practitioner can then engage the band around the organ and then bring the ends closer to each other so as to make them touch each other and form a closed loop around said organ. The engagement of the locking means 5,6 hold the ring in a closed configuration.

When the practitioner brings the two ends 3, 4 of the flexible band close to each other, he forces the flexion of said flexible band 2, which causes the folds formed by the ridges to be further pronounced and therefore gradually closes the angle formed between consecutive facets.

Advantageously, the folds thus formed are spontaneously localized in a substantially uniform angular distribution at the transition zones which form features of incipient deformation under flexion.

Once the ring is in place, the facets come into contact with the wall of the biological organ.

The practitioner can then introduce a fluid under pressure into the inflatable pouch 30 and thus bring about the deformation of the facets 20, 120, 220, 320.

More particularly, he causes said facets, i.e. the intermediate free zones included between the ridges, to bulge so that the facets form domes which well up in a centripetal radial direction, gradually gripping the biological organ.

Advantageously, during said inflating operation, the tie-rods 41 maintain the ridges in a withdrawn position from the facets thus accentuating the folds formed between two neighboring facets and limiting the substantially centripetal elastic deformation to the facets alone and more particularly to the central zone of each facet situated at equal distance from the two ridges which demarcate this facet.

Thus, precise control is achieved over the zones of deformation of the ring 1 as also over the total diameter of the aperture of said ring through which the biological organ passes.

If the practitioner wishes to relax the grip exerted by the ring on the biological organ, or even extract the ring, he can draw fluid from the pouch 30 so as to deflate it.

The domes formed at the facets tend to collapse by elastic return so that the ventral face 10 gradually recovers its structure at rest with substantially flat facets.

If need be, the practitioner can unlock the ring so as to open the loop, for example by making use of the grasping tabs 7, 8.

Thus, the ring 1 of the invention provides an adaptable, non-traumatic gripping quality which conforms to the physical integrity of the ring since it limits the work under fatigue of the membrane 31 in distributing the folds along the ventral face 10 in a balanced, predictable and controlled way.

In particular, the use of the facets 20, 120, 220, 320 and advantageously of the retaining means 40 makes it possible to maintain a homogenous and circular gripping shape capable of preventing the appearance of pinching zones and/or pressure points traumatic for the biological tissues.

Furthermore, such a ring has a particularly simple structure with a relatively modest manufacturing cost.

Finally, it can be noted that whatever the number of cycles of opening and closing said ring 1 or of inflation/deflation of this ring, its behavior is controlled and reproducible in terms of position, orientation or intensity of the folds formed as well as in terms of expansion or contraction of the facets.

1. Implantable surgical ring configured to be placed around a biological organ constituting a pouch or a conduit in order to modify the section of passage of said biological organ, said ring comprising a flexible band that is capable of getting wound around an axis to form a loop around the organ and that has a ventral face designed to come into contact with said organ, wherein said flexible band is preformed in such a way that the ventral face is curved around said axis and divided into a plurality of contiguous and secant facets which are connected to one another by transition zones forming predefined hinging folds whose angular coverage relatively to the axis is smaller than or equal to 5° each facet covering an angular sector ranging substantially from 15° to 35°.

2. Surgical ring according to claim 1 wherein the transition zones are formed by ridges which correspond to the intersection of the contiguous facets.

3. Surgical ring according to claim 1 wherein the facets demarcate one or more inflatable pouches.

4. Surgical ring according to claim 1 wherein the facets, are mounted so as to be radially mobile relatively to a dorsal portion of the flexible band.

5. Surgical ring according to claim 4 further comprising a retainer which acts on the transition zones in order to counter their radial shift when the facets neighboring them are driven in radial shift.

6. Surgical ring according to claim 5 wherein the retainer includes one or more tie-rods which join the ridges to the dorsal portion of the flexible band.

7. Surgical ring according to claim 6 wherein each ridge has at least two paired tie-rods each extending on a lateral face of the flexible band on either side of the ridge.

8. Surgical ring according to claim 1 wherein when the surgical ring is at rest, the facets are substantially plane.

9. Surgical ring according to claim 1 wherein the facets are substantially identical to one another.

10. Surgical ring according to claim 1 wherein the surgical ring constitutes a gastric bypass ring.

11. Method for manufacturing an implantable surgical ring to be placed around a biological organ forming a pouch or a conduit in order to modify the section of passage of said biological organ, said method comprising a step (a) for making a flexible band that is capable of getting wound around an axis to form a loop around the organ and having a ventral face designed to come into contact with said organ, said method comprising a step (b) for forming a the ventral face, the step (b) comprising a sub-step (b1) for preforming under flexion during which the ventral face is given a shape curved around said axis and a sub-step (b2) of division during which there are formed, on said ventral face, a plurality of contiguous and secant facets that are connected to one another by transition zones forming predefined hinging folds whose angular coverage relatively to the axis is smaller than or equal to 5°, each facet covering an angular sector ranging substantially from 15° to 35°.

12. Method according to claim 11 characterized in that wherein the sub-step (b2) of division is carried out by contact molding of a polyhedral core having a succession of plane faces corresponding to imprints of the facets.

13. Surgical ring according to claim 1 wherein the predefined hinging folds have an angular coverage relative to the axis that is smaller than or equal to 3°.

14. Surgical ring according to claim 1 wherein the predefined hinging folds have an angular coverage relative to the axis that is smaller than or equal to 1°.

15. Method according to claim 11 wherein the predefined hinging folds have an angular coverage relative to the axis that is smaller than or equal to 3°.

16. Method according to claim 11 wherein the predefined hinging folds have an angular coverage relative to the axis that is smaller than or equal to 1°.
